(19) United States
(12) Patent Application Publication
(10) Pub. No.: US 2026/0240497 A1
(43) Pub. Date: Aug. 20, 2026

(54) ELECTRICALLY CONDUCTIVE TRANSPARENT SENSOR COATING

(71) Applicant: Oura Health Oy, Oulu (FI)

(72) Inventors: Jonathan Sami Watson, San Diego, CA (US); Gary Watts, Oceanside, CA (US)

(21) Appl. No.: 19/055,893

(22) Filed: Feb. 18, 2025

(51) Int. Cl. A61B 5/00; A61B 5/024

(52) U.S. Cl. CPC A61B 5/6826; A61B 5/02427; A61B 2562/0238

(57) ABSTRACT

Methods, systems, and devices for a wearable ring device are described. A wearable ring device may include a transparent lens positioned within an aperture of a housing of the wearable device, where at least a first portion of the transparent lens may be electrically conductive. The wearable ring device may additionally include one or more optical components positioned within the wearable ring device such that light passes into or from the one or more optical components through at least the first portion of the transparent lens. Further, the wearable ring device may include circuitry electrically coupled with the first portion of the transparent lens and configured to generate or detect a current through the first portion of the transparent lens.

### FIELD OF TECHNOLOGY

The following relates to wearable devices and data processing, including an electrically conductive transparent sensor coating.

### BACKGROUND

Some wearable devices may be configured to measure physiological data from users to help the users understand more about their overall physiological health and well-being. For example, a wearable device may include one or more sensors capable of measuring physiological data from the user, such as one or more sets of electrodes that generate a current through a body of the user. However, some wearable devices may be small, such that space available for the one or more sensors may be limited and, in some cases, may not support certain types of sensors, such as the one or more sets of electrodes.

### BRIEF DESCRIPTION OF THE DRAWINGS

FIG. 1 shows an example of a wearable ring device that supports an electrically conductive transparent sensor coating in accordance with aspects of the present disclosure.

FIG. 2 illustrates an example of a system that supports an electrically conductive transparent sensor coating in accordance with aspects of the present disclosure.

FIG. 3 illustrates an example of a system that supports an electrically conductive transparent sensor coating in accordance with aspects of the present disclosure.

FIG. 4 illustrates an example of a flowchart that supports an electrically conductive transparent sensor coating in accordance with aspects of the present disclosure.

### DETAILED DESCRIPTION

In some cases, wearable ring devices may collect physiological data associated with a user via one or more electrodes. For example, a wearable device may support multiple sets of electrodes, including at least a first set of electrodes (e.g., a first set of one or more electrodes) and a second set of electrodes (e.g., a second set of one or more electrodes), where the first set of electrodes and the second set of electrodes are positioned relative to the wearable device such that a user may contact the first set of electrodes and the second set of electrodes and create a signal path (e.g., electrical path) external to the wearable ring device (e.g., through a body of the user) between the first set of electrodes and the second set of electrodes. In such cases, the system may measure (e.g., collect) third physiological data, such as electrocardiogram (ECG or EKG) data, bioimpedance (BioZ) data, electrodermal activity (EDA) data, or any combination thereof, based on the signal path created between the first set of electrodes and the second set of electrodes.

However, in some cases, such as with wearable ring devices, a size of the wearable device may be small (e.g., less than a threshold size), such that the first set of electrodes and the second set of electrodes may occupy a relatively large portion of the wearable device (e.g., as compared to another type of wearable device, as compared to one or more other sensors). In such cases, the wearable device may not be capable of supporting one or more other electrical components (e.g., one or more other sensors) based on a lack of available space due to the first set of electrodes and the second set of electrodes. Alternatively, the wearable ring may not be capable of supporting the first set of electrodes and the second set of electrodes based on a lack of available space due to the one or more other electrical components.

Accordingly, techniques described herein may support a wearable ring device with a transparent coating applied to a lens through which one or more optical sensors may transmit or receive light, where the transparent coating is electrically conductive such that the lens (e.g., the coating on the lens) may function as an electrode. For example, an inner housing of the wearable ring device may include at least one aperture (e.g., cut-out) in which a lens is positioned. The lens may be positioned relative to one or more optical sensors on a printed circuit board (PCB) (e.g., between the inner housing and an outer housing of the wearable ring device), such that the one or more optical sensors may be capable of transmitting light, receiving light, or both, through the lens. In some examples, at least a portion of the lens may be coated in an electrically conductive coating, where the electrically conductive coating is also transparent such that the light transmitted, received, or both, by the one or more optical sensors may also propagate through the electrically conductive coating with minimal impact, if any, to physiological data collected by the one or more optical sensors (e.g., the coating may not distort the light, may distort the light less than a threshold distortion). The electrically conductive coating may additionally contact the PCB, such that an electrical path may be created between the electrically conductive coating and the PCB, enabling the lens coated in the electrically conductive coating to function as an electrode. In other words, the PCB may be capable of generating, detecting, or both, a current through the electrically conductive coating.

In some cases, the electrically conductive coating may contact the PCB via a flange. That is, a flange, or tab, coated in the electrically conductive coating may extend from the lens, from the electrically conductive coating, or both, and may at least partially contact the PCB, creating an electrical contact between the electrically conductive coating and the PCB. Additionally, or alternatively, the PCB may include a conductive pad, such that one or more forces applied to the lens (e.g., and the electrically conductive coating) may create contact pressure between the PCB and the lens (e.g., and thus the electrically conductive coating), creating the electrical contact between the electrically conductive coating and the PCB. Additionally, or alternatively, the portion of the lens that is coated in the electrically conductive coating may include at least a portion of a bottom face of the lens, where the portion of the bottom face of the lens (e.g., and thus the electrically conductive coating) contacts the PCB, creating the electrical contact between the electrically conductive coating and the PCB. Additionally, or alternatively, the lens may be secured to the PCB via an electrically conductive adhesive, where the electrically conductive adhesive also contacts the electrically conductive coating, creating the electrical contact between the electrically conductive coating and the PCB through the electrically conductive adhesive.

Additionally, or alternatively, the lens may include multiple portions coated in the electrically conductive coating, where the multiple portions are electrically isolated from each other, such that each portion may be capable of functioning as a separate electrode. For example, both a first portion of the lens and a second portion of the lens may be coated in the electrically conductive coating, where the first portion of the lens is electrically isolated (e.g., separate from) the second portion of the lens, such that the first portion of the lens may be capable of functioning as a first electrode and the second portion of the lens may be capable of functioning as a second electrode. Additionally, or alternatively, the wearable ring device may include multiple apertures, each with a respective lens coated in the electrically conductive coating, such that each lens is capable of functioning as a separate electrode. For example, the wearable ring device may include a first lens, at a first position relative to the wearable ring device, coated in the electrically conductive coating and a second lens, at a second position relative to the wearable ring device, coated in the electrically conductive coating. In such cases, the first portion may be different than the second portion (e.g., and a threshold distance apart) such that the first lens is electrically isolated from the second lens. Thus, the first lens may be capable of functioning as the first electrode and the second lens may be capable of functioning as the second electrode.

In some other examples, the lens may be made of an electrically conductive material or may be impregnated with the electrically conductive material, such that the lens is capable of functioning as an electrode based on the lens being made of the electrically conductive material or being impregnated with the electrically conductive material.

Aspects of the disclosure are initially described in the context of wearable ring devices. Aspects of the disclosure are further described in the context of systems supporting physiological data collection from users via wearable devices. Aspects of the disclosure are further illustrated by and described with reference to wearable ring device diagrams, system diagrams, and flowcharts that relate to an electrically conductive transparent sensor coating.

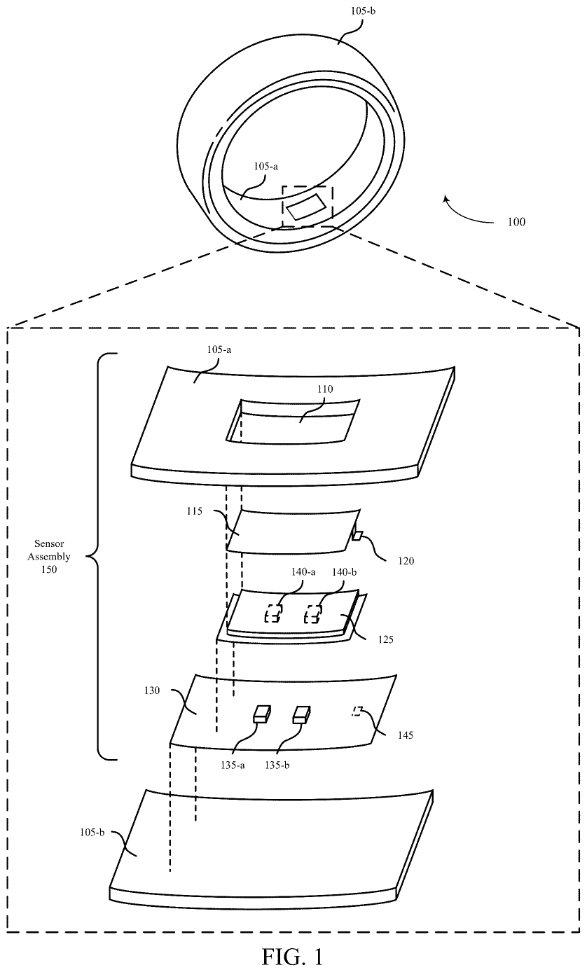

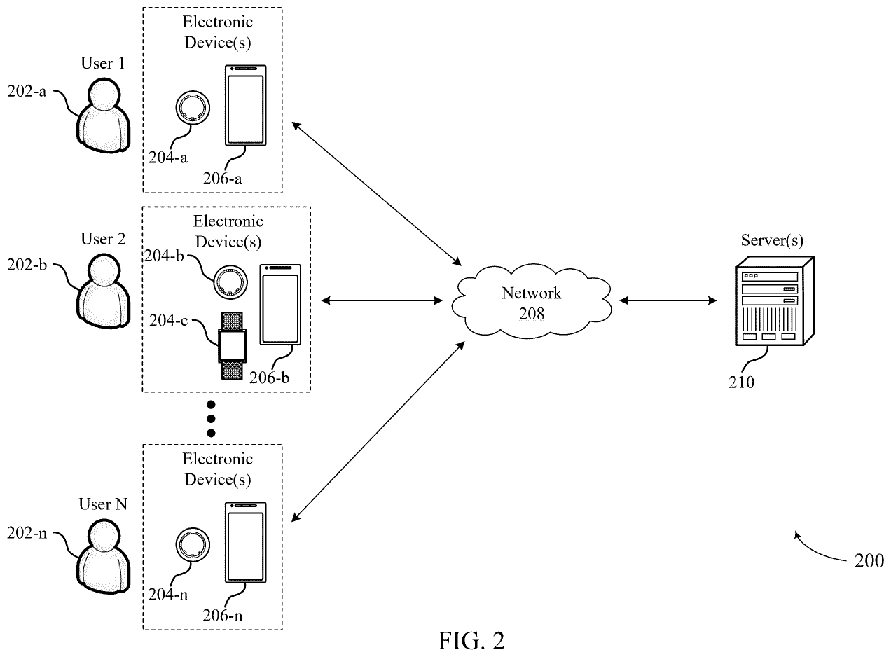

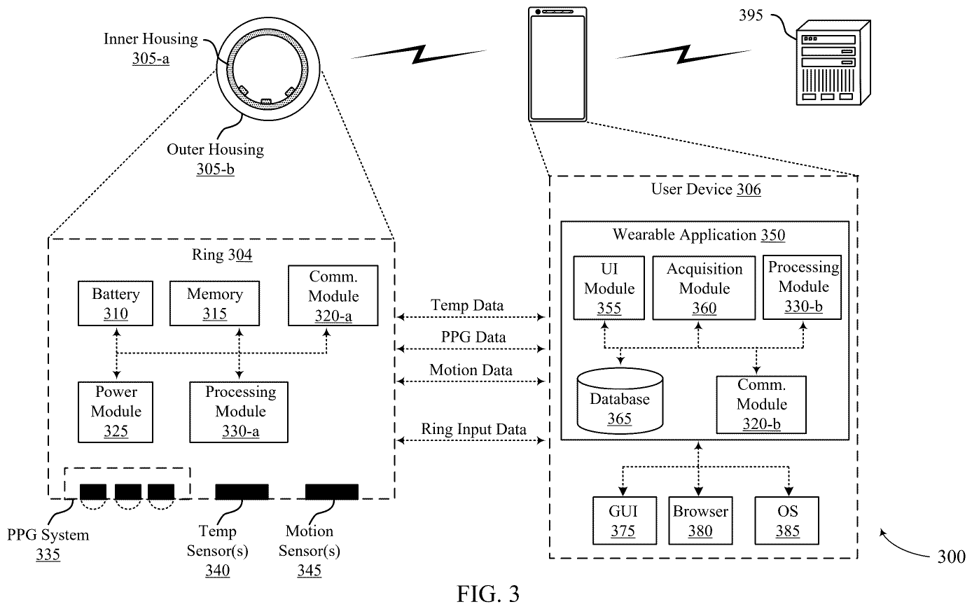

FIG. 1 shows an example of a wearable ring device 100 that supports an electrically conductive transparent sensor coating in accordance with aspects of the present disclosure. The wearable ring device 100 may implement or may be implemented by aspects of a system 200, a system 300, or both, as described with reference to FIGS. 2 and 3. For example, the wearable ring device 100 may be an example of a wearable device 204 or a wearable device 304.

The wearable ring device 100 may include an outer housing 105-b and an inner housing 105-a, where one or more sensors (e.g., one or more optical sensors 135), a PCB 130 (e.g., a flexible PCB 130), one or more other electrical components, or any combination thereof, may be positioned (e.g., at least partially) between the outer housing 105-b and the inner housing 105-a. In other words, the outer housing 105-b and the inner housing 105-a may house (e.g., enclose) the one or more sensors, the PCB 130, the one or more other electrical components, or any combination thereof.

In some cases, the inner housing 105-a (e.g., or the outer housing 105-b) may include an aperture 110 (e.g., a cutout) in which at least a portion of a lens 125 may be positioned. For example, a first portion of the lens 125 may be positioned within (e.g., relative to) the aperture 110 and a second portion of the lens 125 (e.g., a lip, an extension) may be positioned between the inner housing 105-a and the outer housing 105-b, outside of the aperture 110. That is, a surface area of the first portion of the lens 125 may be the same as (e.g., or a threshold deviation smaller than) an area (e.g., surface area) of the aperture 110 (e.g., an opening of the aperture 110), such that the first portion of the lens 125 may fit within the aperture 110. Additionally, a surface area of the second portion of the lens 125 may be larger than the area of the aperture 110, such that the second portion of the lens 125 contacts an internal surface (e.g., facing radially outward) of the inner housing 105-a to secure the lens 125 in place and prevents the lens 125 from falling out of the wearable ring device 100. In some examples, the aperture 110 may extend around a circumference of the inner housing 105-a. In other words, the aperture 110 may include the inner surface of the wearable ring device 100, such that the lens 125 extends around the circumference of the inner housing 105-a (e.g., forming the inner surface).

In such cases, the lens 125 may be transparent (e.g., may be made of a transparent material, such as a polymer or glass) to enable light to propagate through the lens 125. That is, the wearable ring device 100 may additionally include one or more optical sensors 135, such as an optical sensor 135-a and an optical sensor 135-b, positioned between (e.g., relative to) the lens 125 and the PCB 130 (e.g., secured to the PCB 130 underneath the lens 125), such that the one or more optical sensors 135 may transmit light, receive light, or both, through the lens 125. For example, the optical sensor 135-a may be a light emitting component (e.g., a light emitting diode (LED)), such that the optical sensor 135-a transmits light through the lens 125, external to the wearable ring device 100 (e.g., into a finger of the user), and the optical sensor 135-b may be a light detecting component (e.g., photodetector), such that the optical sensor 135-b may receive light through the lens 125.

In some cases, the lens 125 may include one or more cavities 140 (e.g., cutouts, hollow portions) in which the one or more optical sensors 135 may be positioned. For example, a cavity 140-a may align with the optical sensor 135-a and a cavity 140-b may align with the optical sensor 135-b, such that the optical sensor 135-a may be positioned within the cavity 140-a and the optical sensor 135-b may be positioned within the cavity 140-b. In some cases, the one or more cavities 140 may enable the one or more optical sensors 135 to maintain alignment (e.g., be aligned) with the aperture 110.

In some cases, a conductive coating 115 may be applied to (e.g., overlaid on top of, coupled with) at least a portion of the lens 125, such that the lens 125 (e.g., the conductive coating 115) may be capable of functioning as an electrode. In other words, the PCB 130 may be capable of passing a current through the conductive coating 115 and further through a body (e.g., a finger) of a user (e.g., via an electrical, or signal, path external to the wearable ring device 100). In such cases, the conductive coating 115 may additionally be transparent to enable propagation of the light through both the lens 125 (e.g., the portion of the lens 125) and the conductive coating 115. For example, the conductive coating 115 may be made of a transparent material that is also conductive, such as poly(3,4-ethylenedioxythiophene) (PEDOT), indium tin oxide (ITO), or any other transparent conductive material.

In such cases, the conductive coating 115 may be transparent such that light propagated through the conductive coating 115 is distorted (e.g., refracted) by less than a threshold distortion (e.g., is not distorted, or is minimally distorted, as compared to light propagated through the lens 125). That is, a difference between physiological data (e.g., an accuracy of physiological data) collected via light propagation through a lens 125 without the conductive coating 115 and the same physiological data collected via light propagation through a lens 125 with the conductive coating 115 may be less than a threshold deviation. Additionally, or alternatively, the conductive coating 115 may be less than a threshold thickness to enable light propagation through the conductive coating 115 (e.g., with the distortion less than the threshold distortion).

In some examples as depicted in FIG. 1, the portion of the lens 125 may include at least a portion of a top surface of the lens 125 (e.g., radially inward facing surface, contacting the finger of the user). In other words, the conductive coating 115 may be applied to the portion of the top surface of the lens 125. In such cases, one or more mechanical features, such as a flange 120, may extend from the lens 125 (e.g., the conductive coating 115) to enable an electrical path (e.g., an electric coupling) between the conductive coating 115 and the PCB 130. That is, to enable propagation of a current through the conductive coating 115, the conductive coating 115 may be electrically coupled to the PCB 130 (e.g., a contact point 145, or a conductive pad, on the PCB 130) via the flange 120. In such cases, the flange 120 may additionally be conductive based on a material of the flange 120 (e.g., metal), based on application of the conductive coating 115 to the flange 120, or both.

Additionally, or alternatively, the portion of the lens 125 may include both the portion of the top surface of the lens 125 and at least a portion of a bottom surface of the lens (e.g., radially outward facing surface, facing the PCB 130). In other words, the conductive coating 115 may be applied to the portion of the top surface of the lens 125 and the portion of the bottom surface of the lens 125 (e.g., and at least a portion of a side of the lens 125 to connect the conductive coating 115 on the top surface to the conductive coating 115 on the bottom surface). In some examples, a conductive adhesive may be used to secure the lens 125 to the PCB 130, such that the electrical path between the conductive coating 115 and the PCB 130 is via the conductive adhesive. Additionally, or alternatively, the PCB 130 may include a conductive (e.g., metal) pad, such that one or more forces applied to the lens 125 (e.g., and the conductive coating 115) may create contact pressure between the PCB 130 and the lens 125 (e.g., and thus the conductive coating 115), creating the electrical path between the conductive coating 115 and the PCB. Additionally, or alternatively, the lens 125 may be made of a conductive material or may be impregnated with the conductive material, such that the lens 125 itself is conductive.

In some examples, the lens 125 may include multiple portions coated in the conductive coating 115 (e.g., or may include multiple portions made of, or impregnated with, the conductive material), where the multiple portions are electrically (e.g., conductively) isolated from each other, such that each portion may be capable of functioning as a separate electrode. For example, both a first portion of the lens 125 and a second portion of the lens 125 may be coated in the conductive coating 115, where the first portion of the lens 125 is electrically isolated (e.g., separate from) the second portion of the lens 125 such that the first portion of the lens 125 may be capable of functioning as a first electrode and the second portion of the lens 125 may be capable of functioning as a second electrode. In such cases, the first portion of the lens 125 and the second portion of the lens 125 may be separated by a first threshold distance to enable the first portion of the lens 125 and the second portion of the lens 125 to function as different electrodes (e.g., to enable the electrical isolation).

In some cases, the first electrode (e.g., the first portion of the lens 125) and the second electrode (e.g., the second portion of the lens 125) may be part of an electrode pair. That is, the PCB 130 may generate a current that passes through the first electrode, through the finger of the user (e.g., via the electrical path external to the wearable ring device 100), and back through the second electrode (e.g., or visa-versa). Additionally, or alternatively, the first electrode and the second electrode may be part of different electrode pairs. For example, the PCB 130 may generate a first current that passes through the first electrode, through the finger of the user (e.g., via a first electrical path external to the wearable ring device 100), and back through a third electrode and may generate a second current that passes through the second electrode, through the finger of the user (e.g., via a second electrical path external to the wearable ring device 100), and back through a fourth electrode (e.g., the same as or different than the third electrode).

In some cases, the aperture 110, the conductive coating 115 (e.g., and the flange 120), the lens 125, and the one or more optical sensors 135 (e.g., and the one or more cavities 140) may be referred to as a sensor assembly 150, and the wearable ring device 100 may include multiple sensors assemblies 150, where each sensor assembly 150 is capable of functioning as a separate electrode. For example, at a first radial position relative to the inner housing 105-a, the wearable ring device 100 may include a first sensor assembly 150 including a first aperture 110, a first lens 125 coated in the conductive coating 115, and one or more first optical sensors 135 and, at a second radial position relative to the inner housing 105-a, may include a second sensor assembly 150 including a second aperture 110, a second lens 125 coated in the conductive coating 115, and one or more second optical sensors 135. Thus, the first sensor assembly 150 may function as the first electrode and the second sensor assembly 150 may function as the second electrode, such that the PCB 130 may be capable of generating a current via an electrical path between the first electrode and the second electrode, through the finger of the user. In such cases, the first radial position may be different than the second radial position, and the first radial position and the second radial position may be separated by a second threshold distance (e.g., a threshold radial distance).

Thus, as described herein, the wearable ring device 100 may collect physiological data based on one or more currents generated through (e.g., via) the conductive coating 115, the lens 125, or both (e.g., based on the conductive coating 115, the lens 125, or both, functioning as an electrode). Examples of the physiological data may include ECG (e.g., EKG) data, BioZ data, EDA data, or the like thereof. In some examples, a system associated with the wearable ring device 100 may prevent the wearable ring device 100 from collecting first physiological data via the one or more optical sensors 135 and second physiological data via generation of a current through the conductive coating 115, the lens 125, or both, at a same time. That is, the system may activate the one or more optical sensors 135 at a first time to collect the first physiological data and may generate the current via (e.g., through) the conductive coating 115, the lens 125, or both, at a second time to collect the second physiological data, where the first time is different than the second time.

It is to be understood that the conductive coating 110 may be considered a separate component to the lens 125 or may be considered part of the lens 125, such that the phrase “the lens 125 may be capable of functioning as an electrode” is synonymous to “the conductive coating 115 may be capable of functioning as an electrode” and “the lens 125 coated in the conductive coating 115 may be capable of functioning as an electrode.”

Though depicted in the context of the optical sensor 135-a and the optical sensor 135-b, this is not to be regarded as a limitation of the present disclosure. In this regard, any quantity of optical sensors 135 may be supported with regards to the techniques described herein.

Though depicted in the context of a rectangular aperture 110 (e.g., a rectangular lens 125), this is not to be regarded as a limitation of the present disclosure. In this regard, any shape of aperture 110 may be supported with regards to the techniques described herein, including, but not limited to, circular, oval, triangular, square, rectangular, or the like thereof.

Though described in the context of an aperture 110 in the inner housing 105-a, this is not to be regarded as a limitation of the present disclosure. In this regard, the outer housing 105-b may additionally, or alternatively, include an aperture 110 associated with a sensor assembly 150. Additionally, or alternatively, the outer housing 105-b (e.g., and/or the inner housing 105-a) may include one or more electrode components (e.g., a traditional electrode) capable of forming an electrical path (e.g., capable of propagating a current along the electrical path) with a sensor assembly 150. FIG. 2 illustrates an example of a system 200 that supports an electrically conductive transparent sensor coating in accordance with aspects of the present disclosure. The system 200 includes a plurality of electronic devices (e.g., wearable devices 204, user devices 206) that may be worn and/or operated by one or more users 202. The system 200 further includes a network 208 and one or more servers 210.

The electronic devices may include any electronic devices known in the art, including wearable devices 204 (e.g., ring wearable devices, watch wearable devices, etc.), user devices 206 (e.g., smartphones, laptops, tablets). The electronic devices associated with the respective users 202 may include one or more of the following functionalities: 1) measuring physiological data, 2) storing the measured data, 3) processing the data, 4) providing outputs (e.g., via GUIs) to a user 202 based on the processed data, and 5) communicating data with one another and/or other computing devices. Different electronic devices may perform one or more of the functionalities.

Example wearable devices 204 may include wearable computing devices, such as a ring computing device (hereinafter “ring”) configured to be worn on a user's 202 finger, a wrist computing device (e.g., a smart watch, fitness band, or bracelet) configured to be worn on a user's 202 wrist, and/or a head mounted computing device (e.g., glasses/goggles). Wearable devices 204 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 204 may also be attached to, or included in, articles of clothing. For example, wearable devices 204 may be included in pockets and/or pouches on clothing. As another example, wearable device 204 may be clipped and/or pinned to clothing, or may otherwise be maintained within the vicinity of the user 202. Example articles of clothing may include, but are not limited to, hats, shirts, gloves, pants, socks, outerwear (e.g., jackets), and undergarments. In some implementations, wearable devices 204 may be included with other types of devices such as training/sporting devices that are used during physical activity. For example, wearable devices 204 may be attached to, or included in, a bicycle, skis, a tennis racket, a golf club, and/or training weights.

Much of the present disclosure may be described in the context of a ring wearable device 204. Accordingly, the terms “ring,” “wearable device 204,” and like terms, may be used interchangeably, unless noted otherwise herein. However, the use of the term “ring” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 206 may include handheld mobile computing devices, such as smartphones and tablet computing devices. User devices 206 may also include personal computers, such as laptop and desktop computing devices. Other example user devices 206 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 206 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 204, user devices 206) may measure physiological parameters of respective users 202, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 204), mobile device application, or a server computing device may process received physiological data that was measured by other devices.

In some implementations, a user 202 may operate, or may be associated with, multiple electronic devices, some of which may measure physiological parameters and some of which may process the measured physiological parameters. In some implementations, a user 202 may have a ring (e.g., wearable device 204) that measures physiological parameters. The user 202 may also have, or be associated with, a user device 206 (e.g., mobile device, smartphone), where the wearable device 204 and the user device 206 are communicatively coupled to one another. In some cases, the user device 206 may receive data from the wearable device 204 and perform some/all of the calculations described herein. In some implementations, the user device 206 may also measure physiological parameters described herein, such as motion/activity parameters.

For example, as illustrated in FIG. 2, a first user 202-a (User 1) may operate, or may be associated with, a wearable device 204-a (e.g., ring wearable device) and a user device 206-a that may operate as described herein. In this example, the user device 206-a associated with user 202-a may process/store physiological parameters measured by the wearable device 204-a. Comparatively, a second user 202-b (User 2) may be associated with a wearable device 204-b (e.g., ring wearable device), a wrist-worn wearable device 204-c (e.g., watch), and a user device 206-b, where the user device 206-b associated with user 202-b may process/store physiological parameters measured by the ring 204-b and/or the watch 204-c. Moreover, an nth user 202-n (User N) may be associated with an arrangement of electronic devices described herein (e.g., wearable device 204-n, user device 206-n). In some aspects, wearable devices 204 (e.g., ring wearable devices, wrist-worn wearable devices) and other electronic devices may be communicatively coupled to the user devices 206 of the respective users 202 via Bluetooth, Wi-Fi, and other wireless protocols. Moreover, in some cases, the wearable device 204 and the user device 206 may be included within (or make up) the same device. For example, in some cases, the wearable device 204 may be configured to execute an application associated with the wearable device 204, and may be configured to display data via a GUI.

In some implementations, the wearable devices 204 (e.g., ring wearable devices) of the system 200 may be configured to collect physiological data from the respective users 202 based on arterial blood flow within the user's finger. In particular, a wearable device 204 (e.g., ring wearable device) may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 200 may be configured to collect physiological data from the respective users 202 based on blood flow diffused into a microvascular bed of skin with capillaries and arterioles. For example, the system 200 may collect PPG data based on a measured amount of blood diffused into the microvascular system of capillaries and arterioles. In some implementations, the wearable device 204 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the wearable device 204 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a wearable device 204 has been found to exhibit superior performance as compared to wearable devices worn on the wrist, as the wearable device 204 may have greater access to arteries (as compared to capillaries), thereby resulting in stronger signals and more valuable physiological data.

The electronic devices of the system 200 (e.g., user devices 206, wearable devices 204) may be communicatively coupled to one or more servers 210 via wired or wireless communication protocols. For example, as shown in FIG. 2, the electronic devices (e.g., user devices 206) may be communicatively coupled to one or more servers 210 via a network 208. The network 208 may implement transfer control protocol and internet protocol (TCP/IP), such as the Internet, or may implement other network 208 protocols. Network connections between the network 208 and the respective electronic devices may facilitate transport of data via email, web, text messages, mail, or any other appropriate form of interaction within a computer network 208. For example, in some implementations, the ring 204-a associated with the first user 202-a may be communicatively coupled to the user device 206-a, where the user device 206-a is communicatively coupled to the servers 210 via the network 208. In additional or alternative cases, wearable devices 204 (e.g., ring wearable devices, wrist-worn wearable devices) may be directly communicatively coupled to the network 208.

The system 200 may offer an on-demand database service between the user devices 206 and the one or more servers 210. In some cases, the servers 210 may receive data from the user devices 206 via the network 208, and may store and analyze the data. Similarly, the servers 210 may provide data to the user devices 206 via the network 208. In some cases, the servers 210 may be located at one or more data centers. The servers 210 may be used for data storage, management, and processing. In some implementations, the servers 210 may provide a web-based interface to the user device 206 via web browsers.

In some aspects, the system 200 may detect periods of time that a user 202 is asleep, and classify periods of time that the user 202 is asleep into one or more sleep stages (e.g., sleep stage classification). For example, as shown in FIG. 2, User 202-a may be associated with a wearable device 204-a (e.g., ring wearable device) and a user device 206-a. In this example, the wearable device 204-a may collect physiological data associated with the user 202-a, including temperature, heart rate, HRV, respiratory rate, and the like. In some aspects, data collected by the wearable device 204-a may be input to a machine learning classifier, where the machine learning classifier is configured to determine periods of time that the user 202-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 202-a via a GUI of the user device 206-a. Sleep stage classification may be used to provide feedback to a user 202-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 200 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 202-a via the wearable device 204-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 202 to generate tailored, individualized circadian rhythm adjustment models that are specific to each respective user 202.

In some aspects, the system 200 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, a ring 204 of the system 200 may include a transparent coating applied to a lens through which one or more optical sensors may transmit or receive light, where the transparent coating is electrically conductive such that the lens (e.g., the coating on the lens) may be capable of functioning as an electrode. For example, an inner housing of the ring 204 may include at least one aperture (e.g., cut-out) in which a lens is positioned. The lens may be positioned relative to one or more optical sensors on a PCB (e.g., between the inner housing and an outer housing of the ring 204), such that the one or more optical sensors may be capable of transmitting light, receiving light, or both, through the lens. Additionally, at least a portion of the lens may be coated in an electrically conductive coating, such that the light transmitted, received, or both, by the one or more optical sensors may also propagate through electrically the conductive coating. The electrically conductive coating may additionally contact the PCB, such than an electrical path may be created between the electrically conductive coating and the PCB, enabling the lens coated in the electrically conductive coating to function as an electrode. In other words, the PCB may be capable of generating, detecting, or both, a current through the electrically conductive coating.

It should be appreciated by a person skilled in the art that one or more aspects of the disclosure may be implemented in a system 200 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

FIG. 3 illustrates an example of a system 300 that supports an electrically conductive transparent sensor coating in accordance with aspects of the present disclosure. The system 300 may implement, or be implemented by, system 200. In particular, system 300 illustrates an example of a wearable device 204 (e.g., ring wearable device), a user device 206, and a server 210, as described with reference to FIG. 2.

In some aspects, the ring 304 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 300 further includes a user device 306 (e.g., a smartphone) in communication with the ring 304. For example, the ring 304 may be in wireless and/or wired communication with the user device 306. In some implementations, the ring 304 may send measured and processed data (e.g., temperature data, photoplethysmogram (PPG) data, motion/accelerometer data, ring input data, and the like) to the user device 306. The user device 306 may also send data to the ring 304, such as ring 304 firmware/configuration updates. The user device 306 may process data. In some implementations, the user device 306 may transmit data to the server 395 for processing and/or storage.

The ring 304 may include a housing 305 that may include an inner housing 305-a and an outer housing 305-b. In some aspects, the inner housing 305-a, the outer housing 305-b, or both, may include a curved profile/surface. In particular, the housing 305 may exhibit any curved or “circumferential” profile, including a circular profile, an elliptical profile, and the like. Moreover, in some cases, the inner housing 305-a, the outer housing 305-b, or both, may include both curved (e.g., “circumferential”) and flat/planar portions. For the purposes of the present disclosure, the term “circumferential” may be used interchangeably with the term “curved” to refer to circular-shaped, elliptical-shaped, or other curved-shaped profile.

In some aspects, the housing 305 of the ring 304 may store or otherwise include various components of the ring including, but not limited to, device electronics, a power source (e.g., battery 310, and/or capacitor), one or more substrates (e.g., printable circuit boards) that interconnect the device electronics and/or power source, and the like. The device electronics may include device modules (e.g., hardware/software), such as: a processing module 330-a, a memory 315, a communication module 320-a, a power module 325, and the like. The device electronics may also include one or more sensors. Example sensors may include one or more temperature sensors 340, a PPG sensor assembly (e.g., PPG system 335), and one or more motion sensors 345.

The sensors may include associated modules (not illustrated) configured to communicate with the respective components/modules of the ring 304, and generate signals associated with the respective sensors. In some aspects, each of the components/modules of the ring 304 may be communicatively coupled to one another via wired or wireless connections. Moreover, the ring 304 may include additional and/or alternative sensors or other components that are configured to collect physiological data from the user, including light sensors (e.g., LEDs), oximeters, and the like.

The ring 304 shown and described with reference to FIG. 3 is provided solely for illustrative purposes. As such, the ring 304 may include additional or alternative components as those illustrated in FIG. 3. Other rings 304 that provide functionality described herein may be fabricated. For example, rings 304 with fewer components (e.g., sensors) may be fabricated. In a specific example, a ring 304 with a single temperature sensor 340 (or other sensor), a power source, and device electronics configured to read the single temperature sensor 340 (or other sensor) may be fabricated. In another specific example, a temperature sensor 340 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 340 (or other sensor). In other examples, a ring 304 that includes additional sensors and processing functionality may be fabricated.

The housing 305 may include one or more housing 305 components. The housing 305 may include an outer housing 305-b component (e.g., a shell) and an inner housing 305-a component (e.g., a molding). The housing 305 may include additional components (e.g., additional layers) not explicitly illustrated in FIG. 3. For example, in some implementations, the ring 304 may include one or more insulating layers that electrically insulate the device electronics and other conductive materials (e.g., electrical traces) from the outer housing 305-b (e.g., a metal outer housing 305-b). The housing 305 may provide structural support for the device electronics, battery 310, substrate(s), and other components. For example, the housing 305 may protect the device electronics, battery 310, and substrate(s) from mechanical forces, such as pressure and impacts. The housing 305 may also protect the device electronics, battery 310, and substrate(s) from water and/or other chemicals.

The outer housing 305-b may be fabricated from one or more materials. In some implementations, the outer housing 305-b may include a metal, such as titanium, that may provide strength and abrasion resistance at a relatively light weight. The outer housing 305-b may also be fabricated from other materials, such polymers. In some implementations, the outer housing 305-b may be protective as well as decorative.

The inner housing 305-a may be configured to interface with the user's finger. The inner housing 305-a may be formed from a polymer (e.g., a medical grade polymer) or other material. In some implementations, the inner housing 305-a may be transparent. For example, the inner housing 305-a may be transparent to light emitted by the PPG light emitting diodes (LEDs). In some implementations, the inner housing 305-a component may be molded onto the outer housing 305-b. For example, the inner housing 305-a may include a polymer that is molded (e.g., injection molded) to fit into an outer housing 305-b metallic shell.

The ring 304 may include one or more substrates (not illustrated). The device electronics and battery 310 may be included on the one or more substrates. For example, the device electronics and battery 310 may be mounted on one or more substrates. Example substrates may include one or more printed circuit boards (PCBs), such as flexible PCB (e.g., polyimide). In some implementations, the electronics/battery 310 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 310 to the device electronics.

The device electronics, battery 310, and substrates may be arranged in the ring 304 in a variety of ways. In some implementations, one substrate that includes device electronics may be mounted along the bottom of the ring 304 (e.g., the bottom half), such that the sensors (e.g., PPG system 335, temperature sensors 340, motion sensors 345, and other sensors) interface with the underside of the user's finger. In these implementations, the battery 310 may be included along the top portion of the ring 304 (e.g., on another substrate).

The various components/modules of the ring 304 represent functionality (e.g., circuits and other components) that may be included in the ring 304. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 315 (memory module) of the ring 304 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 315 may store any of the data described herein. For example, the memory 315 may be configured to store data (e.g., motion data, temperature data, PPG data) collected by the respective sensors and PPG system 335. Furthermore, memory 315 may include instructions that, when executed by one or more processing circuits, cause the modules to perform various functions attributed to the modules herein. The device electronics of the ring 304 described herein are only example device electronics. As such, the types of electronic components used to implement the device electronics may vary based on design considerations.

The functions attributed to the modules of the ring 304 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 330-a of the ring 304 may include one or more processors (e.g., processing units), microcontrollers, digital signal processors, systems on a chip (SOCs), and/or other processing devices. The processing module 330-a communicates with the modules included in the ring 304. For example, the processing module 330-a may transmit/receive data to/from the modules and other components of the ring 304, such as the sensors. As described herein, the modules may be implemented by various circuit components. Accordingly, the modules may also be referred to as circuits (e.g., a communication circuit and power circuit).

The processing module 330-a may communicate with the memory 315. The memory 315 may include computer-readable instructions that, when executed by the processing module 330-a, cause the processing module 330-a to perform the various functions attributed to the processing module 330-a herein. In some implementations, the processing module 330-a (e.g., a microcontroller) may include additional features associated with other modules, such as communication functionality provided by the communication module 320-a (e.g., an integrated Bluetooth Low Energy transceiver) and/or additional onboard memory 315.

The communication module 320-a may include circuits that provide wireless and/or wired communication with the user device 306 (e.g., communication module 320-b of the user device 306). In some implementations, the communication modules 320-a, 320-b may include wireless communication circuits, such as Bluetooth circuits and/or Wi-Fi circuits. In some implementations, the communication modules 320-a, 320-b can include wired communication circuits, such as Universal Serial Bus (USB) communication circuits. Using the communication module 320-a, the ring 304 and the user device 306 may be configured to communicate with each other. The processing module 330-a of the ring may be configured to transmit/receive data to/from the user device 306 via the communication module 320-a. Example data may include, but is not limited to, motion data, temperature data, pulse waveforms, heart rate data, HRV data, PPG data, and status updates (e.g., charging status, battery charge level, and/or ring 304 configuration settings). The processing module 330-a of the ring may also be configured to receive updates (e.g., software/firmware updates) and data from the user device 306.

The ring 304 may include a battery 310 (e.g., a rechargeable battery 310). An example battery 310 may include a Lithium-Ion or Lithium-Polymer type battery 310, although a variety of battery 310 options are possible. The battery 310 may be wirelessly charged. In some implementations, the ring 304 may include a power source other than the battery 310, such as a capacitor. The power source (e.g., battery 310 or capacitor) may have a curved geometry that matches the curve of the ring 304. In some aspects, a charger or other power source may include additional sensors that may be used to collect data in addition to, or that supplements, data collected by the ring 304 itself. Moreover, a charger or other power source for the ring 304 may function as a user device 306, in which case the charger or other power source for the ring 304 may be configured to receive data from the ring 304, store and/or process data received from the ring 304, and communicate data between the ring 304 and the servers 395.

In some aspects, the ring 304 includes a power module 325 that may control charging of the battery 310. For example, the power module 325 may interface with an external wireless charger that charges the battery 310 when interfaced with the ring 304. The charger may include a datum structure that mates with a ring 304 datum structure to create a specified orientation with the ring 304 during charging. The power module 325 may also regulate voltage(s) of the device electronics, regulate power output to the device electronics, and monitor the state of charge of the battery 310. In some implementations, the battery 310 may include a protection circuit module (PCM) that protects the battery 310 from high current discharge, over voltage during charging, and under voltage during discharge. The power module 325 may also include electro-static discharge (ESD) protection.

The one or more temperature sensors 340 may be electrically coupled to the processing module 330-a. The temperature sensor 340 may be configured to generate a temperature signal (e.g., temperature data) that indicates a temperature read or sensed by the temperature sensor 340. The processing module 330-a may determine a temperature of the user in the location of the temperature sensor 340. For example, in the ring 304, temperature data generated by the temperature sensor 340 may indicate a temperature of a user at the user's finger (e.g., skin temperature). In some implementations, the temperature sensor 340 may contact the user's skin. In other implementations, a portion of the housing 305 (e.g., the inner housing 305-a) may form a barrier (e.g., a thin, thermally conductive barrier) between the temperature sensor 340 and the user's skin. In some implementations, portions of the ring 304 configured to contact the user's finger may have thermally conductive portions and thermally insulative portions. The thermally conductive portions may conduct heat from the user's finger to the temperature sensors 340. The thermally insulative portions may insulate portions of the ring 304 (e.g., the temperature sensor 340) from ambient temperature.

In some implementations, the temperature sensor 340 may generate a digital signal (e.g., temperature data) that the processing module 330-a may use to determine the temperature. As another example, in cases where the temperature sensor 340 includes a passive sensor, the processing module 330-a (or a temperature sensor 340 module) may measure a current/voltage generated by the temperature sensor 340 and determine the temperature based on the measured current/voltage. Example temperature sensors 340 may include a thermistor, such as a negative temperature coefficient (NTC) thermistor, or other types of sensors including resistors, transistors, diodes, and/or other electrical/electronic components.

The processing module 330-a may sample the user's temperature over time. For example, the processing module 330-a may sample the user's temperature according to a sampling rate. An example sampling rate may include one sample per second, although the processing module 330-a may be configured to sample the temperature signal at other sampling rates that are higher or lower than one sample per second. In some implementations, the processing module 330-a may sample the user's temperature continuously throughout the day and night. Sampling at a sufficient rate (e.g., one sample per second) throughout the day may provide sufficient temperature data for analysis described herein.

The processing module 330-a may store the sampled temperature data in memory 315. In some implementations, the processing module 330-a may process the sampled temperature data. For example, the processing module 330-a may determine average temperature values over a period of time. In one example, the processing module 330-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 315 may store the average temperature values over time. In some implementations, the memory 315 may store average temperatures (e.g., one per minute) instead of sampled temperatures in order to conserve memory 315.

The sampling rate, which may be stored in memory 315, may be configurable. In some implementations, the sampling rate may be the same throughout the day and night. In other implementations, the sampling rate may be changed throughout the day/night. In some implementations, the ring 304 may filter/reject temperature readings, such as large spikes in temperature that are not indicative of physiological changes (e.g., a temperature spike from a hot shower). In some implementations, the ring 304 may filter/reject temperature readings that may not be reliable due to other factors, such as excessive motion during exercise (e.g., as indicated by a motion sensor 345).

The ring 304 (e.g., communication module) may transmit the sampled and/or average temperature data to the user device 306 for storage and/or further processing. The user device 306 may transfer the sampled and/or average temperature data to the server 395 for storage and/or further processing.

Although the ring 304 is illustrated as including a single temperature sensor 340, the ring 304 may include multiple temperature sensors 340 in one or more locations, such as arranged along the inner housing 305-a near the user's finger. In some implementations, the temperature sensors 340 may be stand-alone temperature sensors 340. Additionally, or alternatively, one or more temperature sensors 340 may be included with other components (e.g., packaged with other components), such as with the accelerometer and/or processor.

The processing module 330-a may acquire and process data from multiple temperature sensors 340 in a similar manner described with respect to a single temperature sensor 340. For example, the processing module 330 may individually sample, average, and store temperature data from each of the multiple temperature sensors 340. In other examples, the processing module 330-a may sample the sensors at different rates and average/store different values for the different sensors. In some implementations, the processing module 330-a may be configured to determine a single temperature based on the average of two or more temperatures determined by two or more temperature sensors 340 in different locations on the finger.

The temperature sensors 340 on the ring 304 may acquire distal temperatures at the user's finger (e.g., any finger). For example, one or more temperature sensors 340 on the ring 304 may acquire a user's temperature from the underside of a finger or at a different location on the finger. In some implementations, the ring 304 may continuously acquire distal temperature (e.g., at a sampling rate). Although distal temperature measured by a ring 304 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 304 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 304 may include a PPG system 335. The PPG system 335 may include one or more optical transmitters that transmit light. The PPG system 335 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 335 may indicate the perfusion of blood in the illuminated region. For example, the PPG signal may indicate blood volume changes in the illuminated region caused by a user's pulse pressure. The processing module 330-a may sample the PPG signal and determine a user's pulse waveform based on the PPG signal. The processing module 330-a may determine a variety of physiological parameters based on the user's pulse waveform, such as a user's respiratory rate, heart rate, HRV, oxygen saturation, and other circulatory parameters.

In some implementations, the PPG system 335 may be configured as a reflective PPG system 335 where the optical receiver(s) receive transmitted light that is reflected through the region of the user's finger. In some implementations, the PPG system 335 may be configured as a transmissive PPG system 335 where the optical transmitter(s) and optical receiver(s) are arranged opposite to one another, such that light is transmitted directly through a portion of the user's finger to the optical receiver(s).

The number and ratio of transmitters and receivers included in the PPG system 335 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 335.

The PPG system 335 illustrated in FIG. 3 may include a reflective PPG system 335 in some implementations. In these implementations, the PPG system 335 may include a centrally located optical receiver (e.g., at the bottom of the ring 304) and two optical transmitters located on each side of the optical receiver. In this implementation, the PPG system 335 (e.g., optical receiver) may generate the PPG signal based on light received from one or both of the optical transmitters. In other implementations, other placements, combinations, and/or configurations of one or more optical transmitters and/or optical receivers are contemplated.

The processing module 330-a may control one or both of the optical transmitters to transmit light while sampling the PPG signal generated by the optical receiver. In some implementations, the processing module 330-a may cause the optical transmitter with the stronger received signal to transmit light while sampling the PPG signal generated by the optical receiver. For example, the selected optical transmitter may continuously emit light while the PPG signal is sampled at a sampling rate (e.g., 350 Hz).

Sampling the PPG signal generated by the PPG system 335 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 330-a may store the pulse waveform in memory 315 in some implementations. The processing module 330-a may process the pulse waveform as it is generated and/or from memory 315 to determine user physiological parameters described herein.

The processing module 330-a may determine the user's heart rate based on the pulse waveform. For example, the processing module 330-a may determine heart rate (e.g., in beats per minute) based on the time between peaks in the pulse waveform. The time between peaks may be referred to as an interbeat interval (IBI). The processing module 330-a may store the determined heart rate values and IBI values in memory 315.

The processing module 330-a may determine HRV over time. For example, the processing module 330-a may determine HRV based on the variation in the IBIs. The processing module 330-a may store the HRV values over time in the memory 315. Moreover, the processing module 330-a may determine the user's respiratory rate over time. For example, the processing module 330-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 330-a may store user respiratory rate values over time in the memory 315.

The ring 304 may include one or more motion sensors 345, such as one or more accelerometers (e.g., 6-D accelerometers) and/or one or more gyroscopes (gyros). The motion sensors 345 may generate motion signals that indicate motion of the sensors. For example, the ring 304 may include one or more accelerometers that generate acceleration signals that indicate acceleration of the accelerometers. As another example, the ring 304 may include one or more gyro sensors that generate gyro signals that indicate angular motion (e.g., angular velocity) and/or changes in orientation. The motion sensors 345 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 330-a may sample the motion signals at a sampling rate (e.g., 50 Hz) and determine the motion of the ring 304 based on the sampled motion signals. For example, the processing module 330-a may sample acceleration signals to determine acceleration of the ring 304. As another example, the processing module 330-a may sample a gyro signal to determine angular motion. In some implementations, the processing module 330-a may store motion data in memory 315. Motion data may include sampled motion data as well as motion data that is calculated based on the sampled motion signals (e.g., acceleration and angular values).

The ring 304 may store a variety of data described herein. For example, the ring 304 may store temperature data, such as raw sampled temperature data and calculated temperature data (e.g., average temperatures). As another example, the ring 304 may store PPG signal data, such as pulse waveforms and data calculated based on the pulse waveforms (e.g., heart rate values, IBI values, HRV values, and respiratory rate values). The ring 304 may also store motion data, such as sampled motion data that indicates linear and angular motion.

The ring 304, or other computing device, may calculate and store additional values based on the sampled/calculated physiological data. For example, the processing module 330 may calculate and store various metrics, such as sleep metrics (e.g., a Sleep Score), activity metrics, and readiness metrics. In some implementations, additional values/metrics may be referred to as “derived values.” The ring 304, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 304 is oriented on the user's finger and if the ring 304 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 330-a may compress the data stored in memory 315. For example, the processing module 330-a may delete sampled data after making calculations based on the sampled data. As another example, the processing module 330-a may average data over longer periods of time in order to reduce the number of stored values. In a specific example, if average temperatures for a user over one minute are stored in memory 315, the processing module 330-a may calculate average temperatures over a five minute time period for storage, and then subsequently erase the one minute average temperature data. The processing module 330-a may compress data based on a variety of factors, such as the total amount of used/available memory 315 and/or an elapsed time since the ring 304 last transmitted the data to the user device 306.

Although a user's physiological parameters may be measured by sensors included on a ring 304, other devices may measure a user's physiological parameters.

For example, although a user's temperature may be measured by a temperature sensor 340 included in a ring 304, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 304 can make physiological measurements in a resting/sleep state in order to acquire cleaner physiological signals. In one example, the ring 304 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 304 may be configured to collect, store, and/or process data, and may transfer any of the data described herein to the user device 306 for storage and/or processing. In some aspects, the user device 306 includes a wearable application 350, an operating system (OS) 385, a web browser application (e.g., web browser 380), one or more additional applications, and a GUI 375. The user device 306 may further include other modules and components, including sensors, audio devices, haptic feedback devices, and the like. The wearable application 350 may include an example of an application (e.g., “app”) that may be installed on the user device 306. The wearable application 350 may be configured to acquire data from the ring 304, store the acquired data, and process the acquired data as described herein. For example, the wearable application 350 may include a user interface (UI) module 355, an acquisition module 360, a processing module 330-b, a communication module 320-b, and a storage module (e.g., database 365) configured to store application data.

In some cases, the wearable device 304 and the user device 306 may be included within (or make up) the same device. For example, in some cases, the wearable device 304 may be configured to execute the wearable application 350, and may be configured to display data via the GUI 375.

The various data processing operations described herein may be performed by the ring 304, the user device 306, the servers 395, or any combination thereof. For example, in some cases, data collected by the ring 304 may be pre-processed and transmitted to the user device 306. In this example, the user device 306 may perform some data processing operations on the received data, may transmit the data to the servers 395 for data processing, or both. For instance, in some cases, the user device 306 may perform processing operations that require relatively low processing power and/or operations that require a relatively low latency, whereas the user device 306 may transmit the data to the servers 395 for processing operations that require relatively high processing power and/or operations that may allow relatively higher latency.

In some aspects, the ring 304, user device 306, and server 395 of the system 300 may be configured to evaluate sleep patterns for a user. In particular, the respective components of the system 300 may be used to collect data from a user via the ring 304, and generate one or more scores (e.g., Sleep Score, Readiness Score) for the user based on the collected data. For example, as noted previously herein, the ring 304 of the system 300 may be worn by a user to collect data from the user, including temperature, heart rate, HRV, and the like. Data collected by the ring 304 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 304 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 300 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 300 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the ring 304 of the system 300 may include a transparent coating applied to a lens through which one or more optical sensors (e.g., of the PPG system 335) may transmit or receive light, where the transparent coating is electrically conductive such that the lens (e.g., the coating on the lens) may be capable of functioning as an electrode. For example, the inner housing 305-a of the ring 304 may include at least one aperture (e.g., cut-out) in which a lens is positioned. The lens may be positioned relative to one or more optical sensors on a PCB (e.g., between the inner housing 305-a and the outer housing 305-b), such that the one or more optical sensors may be capable of transmitting light, receiving light, or both, through the lens. Additionally, at least a portion of the lens may be coated in an electrically conductive coating, such that the light transmitted, received, or both, by the one or more optical sensors may also propagate through the electrically conductive coating. The electrically conductive coating may additionally contact the PCB, such that an electrical path may be created between the electrically conductive coating and the PCB, enabling the lens coated in the electrically conductive coating to function as an electrode. In other words, the PCB may be capable of generating, detecting, or both, a current through the electrically conductive coating.

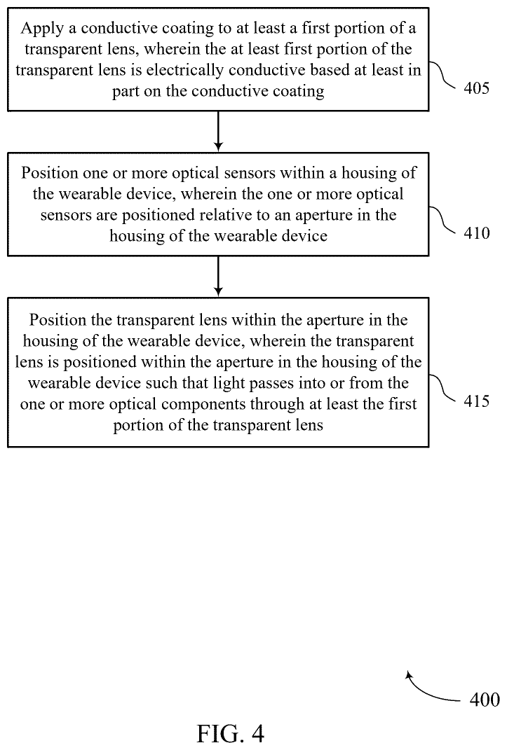

FIG. 4 shows a flowchart illustrating a method 400 that supports electrically conductive transparent sensor coating in accordance with aspects of the present disclosure. The operations of the method 400 may be implemented by a manufacturing system or one or more controllers associated with a manufacturing system. In some examples, one or more controllers may execute a set of instructions to control one or more functional elements of the manufacturing system to perform the described functions. Additionally, or alternatively, one or more controllers may perform aspects of the described functions using special-purpose hardware.

At 405, the method may include applying a conductive coating to at least a first portion of a transparent lens, wherein the at least first portion of the transparent lens is electrically conductive based at least in part on the conductive coating. The operations of 405 may be performed in accordance with examples as disclosed herein.

At 410, the method may include positioning one or more optical sensors within a housing of the wearable device, wherein the one or more optical sensors are positioned relative to an aperture in the housing of the wearable device. The operations of 410 may be performed in accordance with examples as disclosed herein.

At 415, the method may include positioning the transparent lens within the aperture in the housing of the wearable device, wherein the transparent lens is positioned within the aperture in the housing of the wearable device such that light passes into or from the one or more optical components through at least the first portion of the transparent lens. The operations of 415 may be performed in accordance with examples as disclosed herein.

It should be noted that the methods described above describe possible implementations, and that the operations and the steps may be rearranged or otherwise modified and that other implementations are possible. Furthermore, aspects from two or more of the methods may be combined.

A wearable device is described. The wearable device may include a transparent lens positioned within an aperture of a housing of the wearable device, wherein at least a first portion of the transparent lens is electrically conductive, one or more optical components positioned within the wearable device such that light passes into or from the one or more optical components through at least the first portion of the transparent lens, and circuitry electrically coupled with the first portion of the transparent lens and configured to generate or detect a current through the first portion of the transparent lens.

Some examples of the wearable device may further include a conductive coating applied to the first portion of the transparent lens, wherein the first portion of the transparent lens may be electrically conductive based at least in part on the conductive coating.

Some examples of the wearable device may further include a flexible printed circuit board coupled with the circuitry and a flange extending from the conductive coating and at least partially contacting the flexible printed circuit board, wherein the circuitry may be electrically coupled with the first portion of the transparent lens based at least in part on the flange at least partially contacting the flexible printed circuit board.

Some examples of the wearable device may further include a flexible printed circuit board coupled with the circuitry and comprising a conductive pad, wherein the circuitry may be electrically coupled with the conductive coating based at least in part on the conductive coating at least partially contacting the conductive pad.

In some examples of the wearable device, the wearable device further comprises a flexible printed circuit board coupled with the circuitry, wherein the circuitry may be electrically coupled with the conductive coating based at least in part on the conductive coating applied to the bottom surface of the transparent lens a least partially contacting the flexible printed circuit board.

In some examples of the wearable device, the transparent lens may be attached to the flexible printed circuit board using a conductive adhesive material.

In some examples of the wearable device, a material of the conductive coating may be poly(3,4-ethylenedioxythiophene) (PEDOT) or indium tin oxide (ITO).

In some examples of the wearable device, the light passes into or from the one or more optical components through the first portion of the transparent lens based at least in part on the conductive coating being transparent.

In some examples of the wearable device, at least the first portion of the transparent lens may be impregnated with an electrically conductive material and the first portion of the transparent lens may be electrically conductive based at least in part on the first portion of the transparent lens being impregnated with the electrically conductive material.

In some examples of the wearable device, the first portion of the transparent lens comprises an electrically conductive material and the first portion of the transparent lens may be electrically conductive based at least in part on the first portion of the transparent lens comprising the electrically conductive material.

In some examples of the wearable device, a second portion of the transparent lens may be electrically conductive and may be electrically isolated from the first portion of the transparent lens and the circuitry may be electrically coupled with the second portion of the transparent lens and configured to generate or detect a second current through the second portion of the transparent lens.

In some examples of the wearable device, the circuitry may be configured to generate the current between the first portion of the transparent lens and the second portion of the transparent lens via an electrical path external to the wearable device.

Some examples of the wearable device may further include a second transparent lens positioned within a second aperture of the housing of the wearable device, wherein at least a first portion of the second transparent lens may be electrically conductive, and wherein the circuitry may be electrically coupled with the first portion of the second transparent lens and configured to generate or detect the current through the first portion of the transparent lens and one or more second optical components positioned within the wearable device such that light passes into or from the one or more second optical components through at least the first portion of the second transparent lens.

In some examples of the wearable device, the circuitry may be configured to generate the current between the first portion of the transparent lens and the first portion of the second transparent lens via an electrical path external to the wearable device.

In some examples of the wearable device, the transparent lens may be located at a first radial position, the second transparent lens may be located at a second radial position, and a distance between the first radial position and the second radial position satisfies a threshold.

In some examples of the wearable device, the aperture of the housing of the wearable device comprises an inner surface of the wearable device.

In some examples of the wearable device, the transparent lens comprises one or more cavities configured to house the one or more optical components.

In some examples of the wearable device, the one or more optical components comprise one or more light detecting components, one or more light emitting components, or both.

In some examples of the wearable device, the one or more optical components may be configured to acquire physiological data from a user.

In some examples of the wearable device, the wearable device comprises a wearable ring device.

A method of manufacturing a wearable device is described. The method may include applying a conductive coating to at least a first portion of a transparent lens, wherein the at least first portion of the transparent lens is electrically conductive based at least in part on the conductive coating, positioning one or more optical sensors within a housing of the wearable device, wherein the one or more optical sensors are positioned relative to an aperture in the housing of the wearable device, and positioning the transparent lens within the aperture in the housing of the wearable device, wherein the transparent lens is positioned within the aperture in the housing of the wearable device such that light passes into or from the one or more optical components through at least the first portion of the transparent lens.

Some examples of the method described herein may further include operations, features, means, or instructions for positioning a flexible printed circuit board within the housing of the wearable device, wherein a flange extends from the conductive coating and at least partially contacts the flexible printed circuit board.

Some examples of the method described herein may further include operations, features, means, or instructions for positioning a flexible printed circuit board within the housing of the wearable device, the flexible printed circuit board comprising a conductive pad, wherein the conductive coating at least partially contacts the conductive pad.

In some examples of the method described herein, applying the conductive coating to the at least first portion of the transparent lens may include operations, features, means, or instructions for applying the conductive coating to a top surface of the transparent lens and a bottom surface of the transparent lens.

Some examples of the method described herein may further include operations, features, means, or instructions for positioning a flexible printed circuit board within the housing of the wearable device, wherein the conductive coating applied to the bottom surface of the transparent lens at least partially contacts the flexible printed circuit board.

Some examples of the method described herein may further include operations, features, means, or instructions for attaching the transparent lens to the flexible printed circuit board using a conductive adhesive material.

In some examples of the method described herein, a material of the conductive coating may be poly(3,4-ethylenedioxythiophene) (PEDOT) or indium tin oxide (ITO).

Some examples of the method described herein may further include operations, features, means, or instructions for applying a second conductive coating to at least a first portion of a second transparent lens, wherein the at least first portion of the second transparent lens may be electrically conductive based at least in part on the conductive coating, positioning one or more second optical sensors within the housing of the wearable device, wherein the one or more second optical sensors may be positioned relative to a second aperture in the housing of the wearable device, and positioning the second transparent lens within the second aperture in the housing of the wearable device, wherein the second transparent lens may be positioned within the second aperture in the housing of the wearable device such that light passes into or from the one or more second optical components through at least the first portion of the second transparent lens.

In some examples of the method described herein, the transparent lens may be located at a first radial position, the second transparent lens may be located at a second radial position, and a distance between the first radial position and the second radial position satisfies a threshold.

In some examples of the method described herein, the wearable device comprises a wearable ring device.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

1. A wearable device, comprising:
a transparent lens positioned within an aperture of a housing of the wearable device, wherein at least a first portion of the transparent lens is electrically conductive;
one or more optical components positioned within the wearable device such that light passes into or from the one or more optical components through at least the first portion of the transparent lens; and
circuitry electrically coupled with the first portion of the transparent lens and configured to generate or detect a current through the first portion of the transparent lens.

2. The wearable device of claim 1, further comprising:
a conductive coating applied to the first portion of the transparent lens, wherein the first portion of the transparent lens is electrically conductive based at least in part on the conductive coating.

3. The wearable device of claim 2, further comprising:
a flexible printed circuit board coupled with the circuitry; and
a flange extending from the conductive coating and at least partially contacting the flexible printed circuit board, wherein the circuitry is electrically coupled with the first portion of the transparent lens based at least in part on the flange at least partially contacting the flexible printed circuit board.

4. The wearable device of claim 2, further comprising:
a flexible printed circuit board coupled with the circuitry and comprising a conductive pad, wherein the circuitry is electrically coupled with the conductive coating based at least in part on the conductive coating at least partially contacting the conductive pad.

5. The wearable device of claim 2, wherein the conductive coating is applied to a top surface of the transparent lens and a bottom surface of the transparent lens, and wherein the wearable device further comprises:
a flexible printed circuit board coupled with the circuitry, wherein the circuitry is electrically coupled with the conductive coating based at least in part on the conductive coating applied to the bottom surface of the transparent lens at least partially contacting the flexible printed circuit board.

6. The wearable device of claim 5, wherein the transparent lens is attached to the flexible printed circuit board using a conductive adhesive material.

7. The wearable device of claim 2, wherein a material of the conductive coating is poly(3,4-ethylenedioxythiophene) (PEDOT) or indium tin oxide (ITO).

8. The wearable device of claim 2, wherein the one or more optical components are positioned within the wearable device such that the light passes into or from the one or more optical components through the first portion of the transparent lens based at least in part on the conductive coating being transparent.

9. The wearable device of claim 1, wherein at least the first portion of the transparent lens is impregnated with an electrically conductive material, and wherein the first portion of the transparent lens is electrically conductive based at least in part on the first portion of the transparent lens being impregnated with the electrically conductive material.

10. The wearable device of claim 1, wherein the first portion of the transparent lens comprises an electrically conductive material, and wherein the first portion of the transparent lens is electrically conductive based at least in part on the first portion of the transparent lens comprising the electrically conductive material.

11. The wearable device of claim 1, wherein a second portion of the transparent lens is electrically conductive and is electrically isolated from the first portion of the transparent lens, and wherein the circuitry is electrically coupled with the second portion of the transparent lens and configured to generate or detect a second current through the second portion of the transparent lens.

12. The wearable device of claim 11, wherein the circuitry is configured to generate the current between the first portion of the transparent lens and the second portion of the transparent lens via an electrical path external to the wearable device.

13. The wearable device of claim 1, further comprising:
a second transparent lens positioned within a second aperture of the housing of the wearable device, wherein at least a first portion of the second transparent lens is electrically conductive, and wherein the circuitry is electrically coupled with the first portion of the second transparent lens and configured to generate or detect the current through the first portion of the transparent lens; and
one or more second optical components positioned within the wearable device such that light passes into or from the one or more second optical components through at least the first portion of the second transparent lens.

14. The wearable device of claim 13, wherein the circuitry is configured to generate the current between the first portion of the transparent lens and the first portion of the second transparent lens via an electrical path external to the wearable device.

15. The wearable device of claim 14, wherein the transparent lens is located at a first radial position, wherein the second transparent lens is located at a second radial position, and wherein a distance between the first radial position and the second radial position satisfies a threshold.

16. The wearable device of claim 1, wherein the aperture of the housing of the wearable device comprises an inner surface of the wearable device.

17. The wearable device of claim 1, wherein the transparent lens comprises one or more cavities configured to house the one or more optical components.

18. The wearable device of claim 1, wherein the one or more optical components comprise one or more light detecting components, one or more light emitting components, or both.

19. The wearable device of claim 1, wherein the one or more optical components are configured to acquire physiological data from a user.

20. The wearable device of claim 1, wherein the wearable device comprises a wearable ring device.

21-30. (canceled)
